(19) United States
(12) Patent Application Publication
(10) Pub. No.: US 2003/0195631 A1
(43) Pub. Date: Oct. 16, 2003

(54) Shape-memory spacers for artificial disc replacements

(76) Inventor: Bret A. Ferree, Cincinnati, OH (US)

Correspondence Address: John G. Posa, Gifford, Krass, Groh, Sprinkle, Anderson & Citkowski, P.C., 280 N. Old Woodward Ave., Suite 400, Birmingham, MI, 48009-5394, US

(21) Appl. No.: 10/412,917

(22) Filed: Apr. 14, 2003

Related U.S. Application Data
(60) Provisional application No. 60/372,410, filed on Apr. 12, 2002.

(51) Int. Cl. A61F 2/44

(52) U.S. Cl. 623/17.16

(57) ABSTRACT

Spacers made of shape-memory material are used to ease insertion into an intradiscal space. In the preferred embodiment the spacer assumes a biconvex shape once inside the disc space. Alternatively, however, the may have concave surfaces, convex and concave surfaces, or either a convex or a concave surface. Any suitable material having shape-memory properties may be used, including a metal such as Nitinol or a polymer such as hydrogel or methacrylate, including stearle methacrylate. Thus, the invention is not limited to materials that strictly adhere to the definition of “shape-memory,” in that substances that assume an expanded state due to temperature change, exposure to moisture, or mechanical relaxation may alternatively be used.

### REFERENCE TO RELATED APPLICATION

This application claims priority from U.S. Provisional Patent Application Serial No. 60/372,410, filed Apr. 12, 2002, the entire content of which is incorporated herein by reference.

### FIELD OF THE INVENTION

This invention relates generally to artificial disc replacements (ADRs) and, in particular, to ADRs incorporating biconvex spacers having shape-memory properties.

### BACKGROUND OF THE INVENTION

Eighty-five percent of the population will experience low back pain at some point. Fortunately, the majority of people recover from their back pain with a combination of benign neglect, rest, exercise, medication, physical therapy, or chiropractic care. A small percent of the population will suffer chronic low back pain. The cost of treatment of patients with spinal disorders plus the patient's lost productivity is estimated at 25 to 100 billion dollars annually.

Seven cervical (neck), 12 thoracic, and 5 lumbar (low back) vertebrae form the normal human spine. Intervertebral discs reside between adjacent vertebra with two exceptions. First, the articulation between the first two cervical vertebrae does not contain a disc. Second, a disc lies between the last lumbar vertebra and the sacrum (a portion of the pelvis).

The spine supports the body, and protects the spinal cord and nerves. The vertebrae of the spine are also supported by ligaments, tendons, and muscles which allow movement (flexion, extension, lateral bending, and rotation). Motion between vertebrae occurs through the disc and two facet joints. The disc lies in the front or anterior portion of the spine. The facet joints lie laterally on either side of the posterior portion of the spine.

The human intervertebral disc is an oval to kidney bean shaped structure of variable size depending on the location in the spine. The outer portion of the disc is known as the annulus fibrosis. The annulus is formed of 10 to 60 fibrous bands. The fibers in the bands alternate their direction of orientation by 30 degrees between each band. The orientation serves to control vertebral motion (one half of the bands tighten to check motion when the vertebra above or below the disc are turned in either direction).

The annulus contains the nucleus. The nucleus pulpous serves to transmit and dampen axial loads. A high water content (70-80 percent) assists the nucleus in this function. The water content has a diurnal variation. The nucleus imbibes water while a person lies recumbent. Activity squeezes fluid from the disc. Nuclear material removed from the body and placed into water will imbibe water swelling to several times its normal size. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per micro liter.

Interestingly, the adult disc is the largest avascular structure in the human body. Given the lack of vascularity, the nucleus is not exposed to the body's immune system. Most cells in the nucleus obtain their nutrition and fluid exchange through diffusion from small blood vessels in adjacent vertebra.

The disc changes with aging. As a person ages the water content of the disc falls from approximately 85 percent at birth to 70 percent in the elderly. The ratio of chondroitin sulfate to keratin sulfate decreases with age. The ratio of chondroitin 6 sulfate to chondroitin 4 sulfate increases with age. The distinction between the annulus and the nucleus decreases with age. These changes are known as disc degeneration. Generally disc degeneration is painless.

Premature or accelerated disc degeneration is known as degenerative disc disease. A large portion of patients suffering from chronic low back pain are thought to have this condition. As the disc degenerates, the nucleus and annulus functions are compromised.

The nucleus becomes thinner and less able to handle compression loads. The annulus fibers become redundant as the nucleus shrinks. The redundant annular fibers are less effective in controlling vertebral motion. The disc pathology can result in: 1) bulging of the annulus into the spinal cord or nerves; 2) narrowing of the space between the vertebra where the nerves exit; 3) tears of the annulus as abnormal loads are transmitted to the annulus and the annulus is subjected to excessive motion between vertebra; and 4) disc herniation or extrusion of the nucleus through complete annular tears.

Current surgical treatments of disc degeneration are destructive. One group of procedures removes the nucleus or a portion of the nucleus; lumbar discectomy falls in this category. A second group of procedures destroy nuclear material; Chymopapin (an enzyme) injection, laser discectomy, and thermal therapy (heat treatment to denature proteins) fall in this category. A third group, spinal fusion procedures either remove the disc or the disc's function by connecting two or more vertebra together with bone. These destructive procedures lead to acceleration of disc degeneration. The first two groups of procedures compromise the treated disc. Fusion procedures transmit additional stress to the adjacent discs. The additional stress results in premature disc degeneration of the adjacent discs.

Prosthetic disc replacement offers many advantages. The prosthetic disc attempts to eliminate a patient's pain while preserving the disc's function. Current prosthetic disc implants, however, either replace the nucleus or the nucleus and the annulus. Both types of current procedures remove the degenerated disc component to allow room for the prosthetic component. Although the use of resilient materials has been proposed, the need remains for further improvements in the way in which prosthetic components are incorporated into the disc space, and in materials to ensure strength and longevity. Such improvements are necessary, since the prosthesis may be subjected to 100,000,000 compression cycles over the life of the implant. Biconvex polyethylene spacers are inserted between metal plates in many artificial disc replacement (ADR) designs. However, inserting biconvex discs into concavities between the metal plates can be difficult. As shown in FIGS. 1A through 1C, surgeons must distract the metal endplates to allow insertion of the poly disc spacer.

The use of shape-memory materials and alloys has also been proposed in conjunction with disc augmentation or replacement. In my U.S. Pat. No. 6,419,704, annular flaps are created in the annulus fibrosis, so that a prosthesis or prostheses may also be inserted through one or both of the flaps. In addition, the prosthesis or prostheses may be inserted through the annular window following a procedure to remove a herniated nucleus pulpous. If annular flaps are formed, they may be sewn or sealed closed after insertion of the artificial disc or discs.

The prosthetic disc or discs could restore a collapsed disc space by inflation of the prosthesis or prostheses. The vertebrae may also be distracted to restore normal disc height and aid the insertion of the prosthesis or prostheses, mechanically. A malleable band of flexible plastic, metal or other material may be inserted through the annular flaps as shown, or a material with a shape memory may be beneficial for such purpose.

Although artificial disc replacements involving endplates and spacers, including biconvex spacers have been proposed, none utilize a shape-memory material. Examples of prior art devices are disclosed in U.S. Pat. Nos. 4,759,766; 5,401,269; 5,507,816; 5,556,431; 5,674,296; 5,865,846; 5,888,226; 6,001,130; and 6,146.421. Thus, as with other existing devices, the spacers associated with these ADRs also require relatively excessive distraction by the attending surgeon.

### SUMMARY OF THE INVENTION

Broadly, according to this invention, biconvex spacers made of shape-memory material are used to facilitate spacer insertion. In the preferred embodiment the spacer assumes a biconvex shape once inside the disc space. Alternatively, however, the may have concave surfaces, convex and concave surfaces, or either a convex or a concave surface. Any suitable material having shape-memory properties may be used, including a metal such as Nitinol or a polymer such as hydrogel or methacrylate, including stearle methacrylate. Thus, the invention is not limited to materials that strictly adhere to the definition of “shape-memory,” in that substances that assume an expanded state due to temperature change, exposure to moisture, or mechanical relaxation may alternatively be used.

### BRIEF DESCRIPTION OF THE DRAWINGS

FIGS. 1A through 1C illustrate how surgeons must distract the metal endplates to allow insertion of the poly disc spacer;

FIG. 2A is a drawing that shows how material is inserted with a flattened or otherwise more compact shape;

FIG. 2B shows how the material of FIG. 2A assumes a desired biconvex shape after it is positioned between the metal plates;

FIG. 3 is a sagittal cross section through an ADR with an alternative spacer shape;

FIG. 4 is a sagittal cross section through an ADR with yet a different spacer shape; and

FIG. 5 is a sagittal cross section through an ADR with a further alternative shape of the novel spacer.

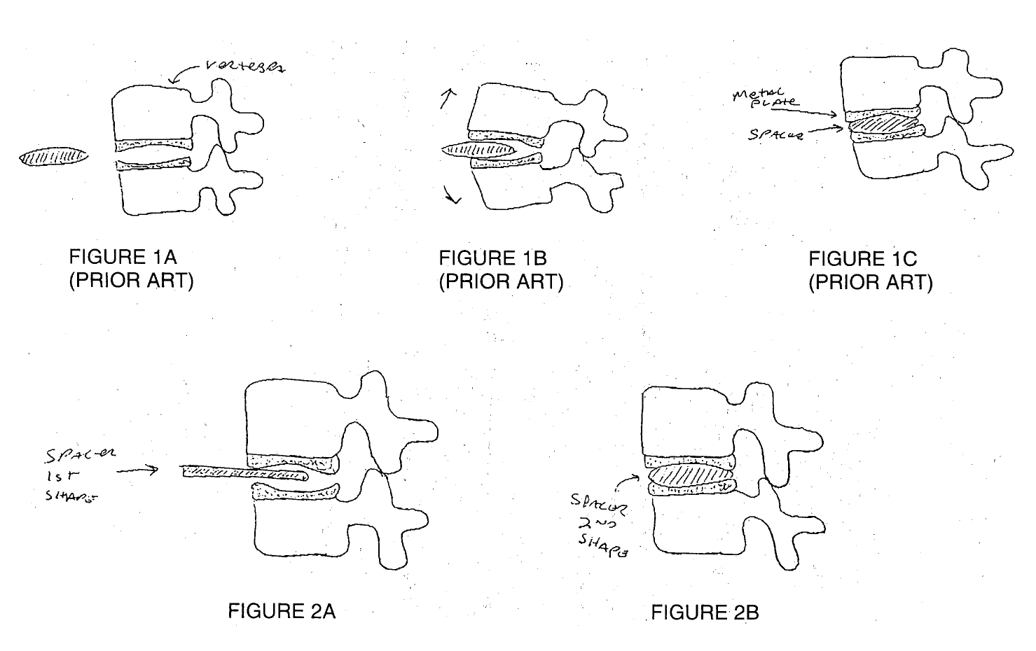

### DETAILED DESCRIPTION OF THE INVENTION

According to this invention, biconvex spacers made of shape memory material are used to facilitate spacer insertion. As shown in FIG. 2A, the material is inserted with a flattened or otherwise more compact shape, but assumes a desired biconvex shape after it is positioned between the metal plates, as shown in FIG. 2B. Any suitable shape-memory material may be used, including metal alloys, polymerics or ceramics, so long as an expansion or inflation is possible after introduction between the opposing plates.

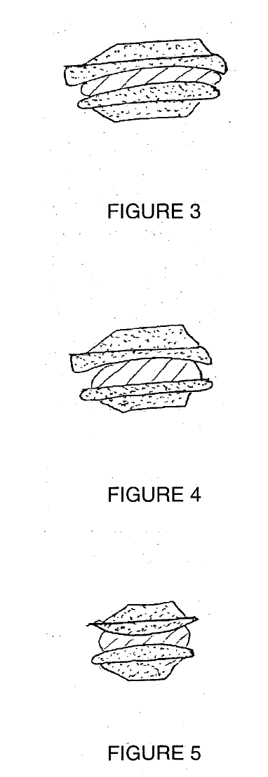

FIG. 3 is a sagittal cross section through an ADR with an alternative shape of a spacer according to the invention. The spacer assumes a shape with convex and concave articulating surfaces.

FIG. 4 is a sagittal cross section through an ADR with another spacer shape. The spacer assumes a shape with convex and straight articulating surfaces. The spacer could also assume a shape with concave and straight articulating surfaces.

FIG. 5 is a sagittal cross section through an ADR with a further alternative spacer shape assumes a shape with two concave articulating surfaces.

I claim:

1. A biconvex spacer adapted to artificial disc replacement surgery, comprising:
a piece of shape memory material having:
a first state which is flattened or otherwise compacted prior to insertion; and
a first state which assumes a desired biconvex shape after it is positioned.
